(19) United States
(12) Patent Application Publication
(10) Pub. No.: US 2026/0130682 A1
(43) Pub. Date: May 14, 2026

(54) TREATMENT TOOL FOR ENDOSCOPE AND ENDOSCOPE SYSTEM

(71) Applicant: FUJIFILM Corporation, Tokyo (JP)

(72) Inventor: Yoshihiro UEDA, Ashigarakami-gun (JP)

(73) Assignee: FUJIFILM Corporation, Tokyo (JP)

(21) Appl. No.: 19/382,270

(22) Filed: Nov. 7, 2025

Related U.S. Application Data
(60) Provisional application No. 63/720,702, filed on Nov. 14, 2024.

(51) Int. Cl. A61B 17/29; A61B 1/018; A61B 1/005; A61B 1/008

(52) U.S. Cl. CPC A61B 17/29; A61B 1/018; A61B 1/0052; A61B 1/008; A61B 2017/2905; A61B 2017/2906

(57) ABSTRACT

A treatment tool for an endoscope, includes: an insertion part; and an operation part disposed on a base end side of the insertion part, the insertion part includes a distal end part having a grip part being openable and closable, a bendable part being bendable and provided adjacent to the distal end part on a side of the operation part, and a connecting part connecting the bendable part and the operation part, the insertion part is capable of being moved forward and backward along a longitudinal axis of the connecting part and rotated about the longitudinal axis by an operation of the operation part, by an operation of the operation part, the grip part is closed and the bendable part is bent in a state where the grip part is closed, and a maximum bending angle of the bendable part to the longitudinal axis is greater than 90 degrees.

### CROSS REFERENCE TO RELATED APPLICATION

This application is based on and claims priority from US Provisional Application No. 63/720,702 filed on November 14, 2024, the entire disclosure of which is incorporated herein by reference.

### BACKGROUND OF THE INVENTION

### 1. Field of the Invention

The present invention relates to a treatment tool for an endoscope and an endoscope system.

### 2. Description of the Related Art

JP1999-299793A (JP-H11-299793A) discloses a subject insertion tool in which a bendable member having a hollow tubular shape and an opening portion on a side is disposed inside a distal end part of a hollow coil that forms an insertion part, a pulling wire is inserted through the hollow coil so as to be movable forward and backward, the pulling wire is biased to an opening portion side of the bendable member and fixed to a distal end side of the bendable member, and the bendable member is bent in an opening portion direction by a pulling operation.

JP1997-299323A (JP-H9-299323A) discloses a treatment tool guide of an endoscope, which is a flexible tubular member that is insertable and removable through a treatment tool insertion channel of the endoscope, and in which a portion protruding outward from a distal end of the treatment tool insertion channel is formed to be bent in an arc shape.

JP2007-151595A discloses an endoscope treatment device provided with an observation unit that observes a body cavity and used in combination with an endoscope that is insertable into the cavity, the endoscope treatment device comprising a flexible insertion part having a bendable part and a channel through which a treatment tool is inserted, an operation part connected to the insertion part and used to operate bending of the bendable part, and a treatment tool operation unit that operates the treatment tool.

JP2022-152489A discloses an endoscope in which an observation window is disposed across a vertical axis in a distal end surface region located above a horizontal axis, a first treatment tool outlet port is disposed across the vertical axis in a distal end surface region located below the horizontal axis, a second treatment tool outlet port is disposed across the horizontal axis in a distal end surface region located below a line which passes through a center of the observation window and which is parallel to the horizontal axis and in a distal end surface region located on a left side of the vertical axis, and a water supply port is mainly disposed in the distal end surface region located below the line which passes through the center of the observation window and which is parallel to the horizontal axis and in a distal end surface region located on a side opposite to the second treatment tool outlet port with respect to a line which passes through a center of the first treatment tool outlet port and which is parallel to the vertical axis.

### SUMMARY OF THE INVENTION

One embodiment according to the technology of the present disclosure provides a treatment tool for an endoscope and an endoscope system capable of improving ease of treatment on a target part.

### (1)

A treatment tool for an endoscope, comprising:

an insertion part that is insertable into a body; and

an operation part that is disposed on a base end side of the insertion part,

in which the insertion part includes

a distal end part having a grip part that is openable and closable,

a bendable part that is bendable and that is provided to be adjacent to the distal end part on a side of the operation part, and

a connecting part that connects the bendable part and the operation part to each other,

the insertion part is capable of being moved forward and backward along a longitudinal axis of the connecting part and being rotated about the longitudinal axis by an operation of the operation part,

by an operation of the operation part, the grip part is closed and the bendable part is bent in a state where the grip part is closed, and

a maximum bending angle of the bendable part with respect to the longitudinal axis is greater than 90 degrees.

### (2)

The treatment tool for an endoscope according to (1), further comprising:

at least one operation wire that is connected to the operation part and that operates at least one of opening and closing of the grip part or bending of the bendable part.

### (3)

The treatment tool for an endoscope according to (1) or (2),

in which the insertion part is used by being inserted into a treatment tool channel of the endoscope, and

an inner diameter of an arc drawn in a case where the bendable part is maximally bent is greater than 1/2 of an outer diameter of the endoscope.

### (4)

The treatment tool for an endoscope according to any one of (1) to (3),

in which the insertion part is used by being inserted into a treatment tool channel of the endoscope, and

in a case where the bendable part is maximally bent, the distal end part extending from the treatment tool channel is not in contact with the endoscope.

### (5)

The treatment tool for an endoscope according to (4),

in which, in a case where the bendable part is maximally bent, the distal end part extending from the treatment tool channel is not in contact with the endoscope regardless of a rotation state of the insertion part.

### (6)

An endoscope system comprising:

the treatment tool for an endoscope according to any one of (1) to (5); and

an endoscope having a treatment tool channel through which the insertion part is insertable.

### (7)

The endoscope system according to (6), further comprising:

a first treatment tool that is the treatment tool for an endoscope;

a second treatment tool; and

the endoscope,

in which the endoscope has a first treatment tool channel through which the insertion part of the first treatment tool is insertable and a second treatment tool channel through which the second treatment tool is insertable.

According to the present invention, it is possible to provide a treatment tool for an endoscope and an endoscope system capable of improving ease of treatment on a target part.

### BRIEF DESCRIPTION OF THE DRAWINGS

FIG. 1 is a diagram showing an example of an endoscope system according to an embodiment of the present invention.

FIG. 2 is a diagram showing an example of a treatment tool for an endoscope 20 according to the embodiment of the present invention.

FIG. 3 is a diagram showing a configuration of a grip part 24 of a distal end part 23 in the treatment tool for an endoscope 20.

FIG. 4 is a diagram showing an operation of the grip part 24 of FIG. 3.

FIG. 5 is a diagram showing a configuration of a bendable part 25 and a connecting part 26 of the treatment tool for an endoscope 20.

FIG. 6 is a diagram showing an operation of the bendable part 25 of FIG. 5.

FIG. 7 is a diagram showing an internal configuration of the bendable part 25.

FIG. 8 is a cross-sectional view taken along line a-a of the bendable part 25 shown in FIG. 7.

FIG. 9 is a diagram showing an internal operation of the bendable part 25.

FIG. 10 is a diagram showing a configuration of an operation part 22 of the treatment tool for an endoscope 20.

FIG. 11 is a cross-sectional view of the operation part 22 of the treatment tool for an endoscope 20.

FIG. 12 is a cross-sectional view showing a closed state of the operation part 22 of the treatment tool for an endoscope 20.

FIG. 13 is a diagram showing a first example (part 1) of a treatment method using the treatment tool for an endoscope 20.

FIG. 14 is a diagram showing a first example (part 2) of a treatment method using the treatment tool for an endoscope 20.

FIG. 15 is a diagram showing a first example (part 3) of a treatment method using the treatment tool for an endoscope 20.

FIG. 16 is a diagram showing a first example (part 1) of a treatment method using a combination of the treatment tool for an endoscope 20 and another treatment tool for an endoscope.

FIG. 17 is a diagram showing a first example (part 2) of a treatment method using a combination of the treatment tool for an endoscope 20 and another treatment tool for an endoscope.

FIG. 18 is a diagram showing a first example (part 3) of a treatment method using a combination of the treatment tool for an endoscope 20 and another treatment tool for an endoscope.

FIG. 19 is a diagram showing a state in which the treatment tool for an endoscope 20 is attached to a first treatment tool insertion port 13 of an endoscope operation part 7.

FIG. 20 is a diagram showing the bendable part 25 in an insertion part 21 in a non-bent state.

FIG. 21 is a perspective view showing the bendable part 25 in a bent state.

FIG. 22 is a plan view of the bendable part 25 shown in FIG. 21.

FIG. 23 is a diagram showing a second example (part 1) of a treatment method using the treatment tool for an endoscope 20.

FIG. 24 is a diagram showing a second example (part 2) of a treatment method using the treatment tool for an endoscope 20.

FIG. 25 is a diagram showing a second example (part 3) of a treatment method using the treatment tool for an endoscope 20.

FIG. 26 is a diagram showing a positional relationship between the distal end part 23 and an endoscope 2 in a case where the bendable part 25 is maximally bent.

FIG. 27 is a diagram showing an example of an end surface 10a of an endoscope distal end part 10.

FIG. 28 is a diagram showing another example of an operation handle 51.

### DESCRIPTION OF THE PREFERRED EMBODIMENTS

Hereinafter, a treatment tool for an endoscope and an endoscope system according to an embodiment of the present invention will be described with reference to the accompanying drawings.

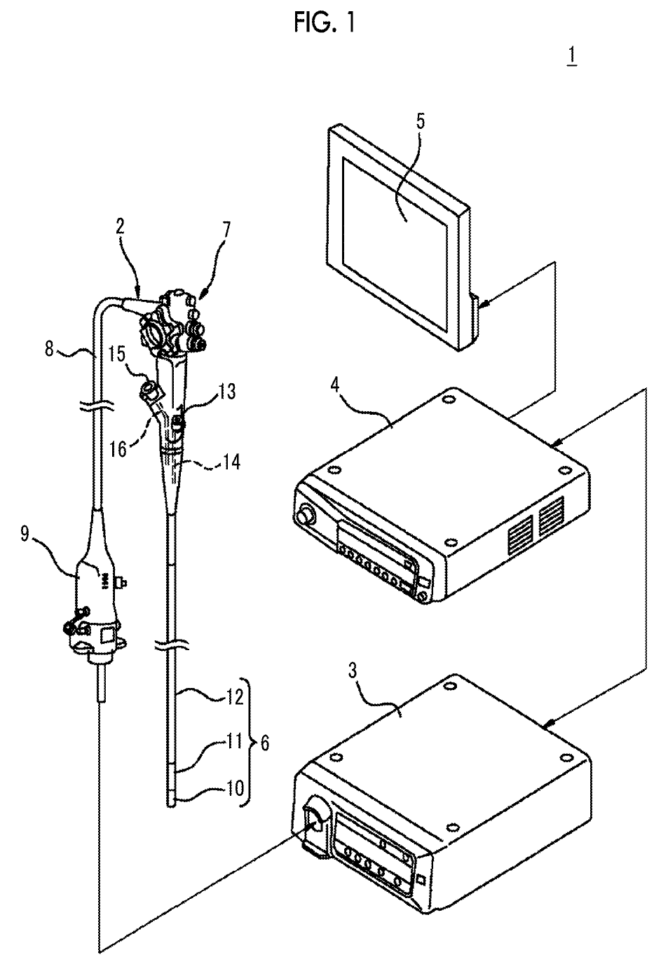

FIG. 1 is a diagram showing an example of an endoscope system according to an embodiment of the present invention. As shown in FIG. 1, an endoscope system 1 comprises an endoscope 2, a light source device 3, and a processor 4. The endoscope 2 has an endoscope insertion part 6 for being inserted into a subject, an endoscope operation part 7 that is connected to the endoscope insertion part 6, and a universal cord 8 that extends from the endoscope operation part 7. The endoscope insertion part 6 is composed of an endoscope distal end part 10, an endoscope bendable part 11 that is connected to the endoscope distal end part 10, and an endoscope connecting part 12 that connects the endoscope bendable part 11 and the endoscope operation part 7.

An imaging apparatus including an imaging element is mounted on the endoscope distal end part 10. The endoscope bendable part 11 is configured to be bendable. The bending of the endoscope bendable part 11 is operated by the endoscope operation part 7. In addition, the endoscope connecting part 12 is configured to be flexible enough to be deformable to follow a shape of an insertion passage in the subject.

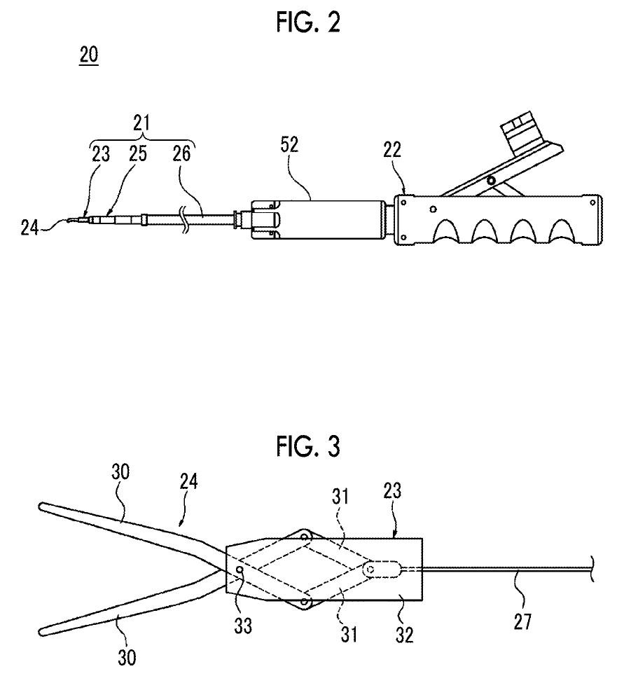

The endoscope operation part 7 is provided with an operation button for operating imaging using the imaging apparatus and an operation knob for operating the bending of the endoscope bendable part 11. In addition, the endoscope operation part 7 is provided with a first treatment tool insertion port 13 into which a treatment tool for an endoscope 20 (see FIG. 2) can be inserted, and a second treatment tool insertion port 15. Inside the endoscope insertion part 6, a first treatment tool channel 14 that reaches the endoscope distal end part 10 from the first treatment tool insertion port 13 and that is open to an end surface of the endoscope distal end part 10, and a second treatment tool channel 16 that reaches the endoscope distal end part 10 from the second treatment tool insertion port 15 and that is open to the end surface of the endoscope distal end part 10 are provided.

A light guide and a cable are provided inside the endoscope insertion part 6, the endoscope operation part 7, and the universal cord 8. A connector 9 is provided at a terminal of the universal cord 8. The endoscope 2 is connected to the light source device 3 and the processor 4 via the connector 9.

Illumination light generated by the light source device 3 is guided to the endoscope distal end part 10 via the light guide and is emitted from the endoscope distal end part 10. In addition, operating power of the imaging element, a control signal for driving the imaging element, and an image signal output from the imaging element are transmitted between the processor 4 and the imaging apparatus via the cable. The processor 4 processes the input image signal to generate image data of an observation site in the subject, displays the generated image data on a monitor 5, and records the generated image data in a storage unit.

FIG. 2 is a view showing an example of the treatment tool for an endoscope 20 according to the embodiment of the present invention. As shown in FIG. 2, the treatment tool for an endoscope 20 comprises an insertion part 21, an operation part 22 of the treatment tool, and a fixing part 52. The insertion part 21 is a portion that can be inserted through the first treatment tool channel 14 (see FIG. 1). The operation part 22 is a portion disposed on a base end side of the insertion part 21. The fixing part 52 is a portion that is attached to and fixed to the endoscope operation part 7.

The insertion part 21 includes a distal end part 23 having a grip part 24 that can be opened and closed by the operation of the operation part 22, a bendable part 25 that is provided adjacent to the operation part 22 side of the distal end part 23, and a connecting part 26 that connects the bendable part 25 and the operation part 22. In a case where the insertion part 21 is inserted through the first treatment tool channel 14, the connecting part 26 is housed in the first treatment tool channel 14. Meanwhile, in a case where the insertion part 21 is inserted through the first treatment tool channel 14, the distal end part 23 and the bendable part 25 protrude from the end surface of the endoscope distal end part 10 (see FIG. 1). As with the endoscope connecting part 12, the connecting part 26 housed in the first treatment tool channel 14 is configured to be flexible enough to be deformable to follow the shape of the insertion passage in the subject.

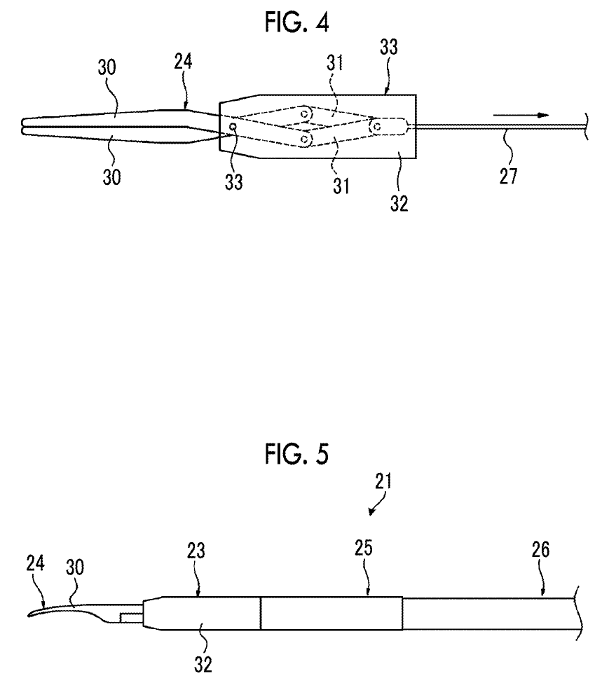

FIG. 3 is a diagram showing a configuration of the grip part 24 of the distal end part 23 in the treatment tool for an endoscope 20. FIG. 4 is a diagram showing an operation of the grip part 24 of FIG. 3.

As shown in FIG. 3, the grip part 24 has a pair of grip claws 30 and a pair of link members 31. The distal end part 23 has a support 32 that rotationally movably supports the pair of grip claws 30. The pair of grip claws 30 are disposed to intersect each other, and a pin 33 is provided to penetrate an intersecting portion of the pair of grip claws 30. The pin 33 is fixed to the support 32. The pair of grip claws 30 are supported by the support 32 so as to be movable rotationally about the pin 33 as a rotational movement shaft.

A distal end part of the link member 31 is rotationally movably connected to a base end part of the grip claw 30. At least one operation wire 27 is connected to a base end part of the link member 31. The operation wire 27 reaches the operation part 22 from the distal end part 23 through the bendable part 25 and the connecting part 26, and is pulled to the operation part 22 side or pushed out to the distal end part 23 side in response to an operation of the operation part 22.

FIG. 3 shows a state where the operation wire 27 is pushed out to the distal end part 23 side, and distal end parts of the pair of grip claws 30 are open. On the other hand, in a case where the operation wire 27 is pulled to the operation part 22 side, the distal end parts of the pair of grip claws 30 are closed as shown in FIG. 4. A treatment site of a living body is gripped by the distal end parts of the pair of closed grip claws 30.

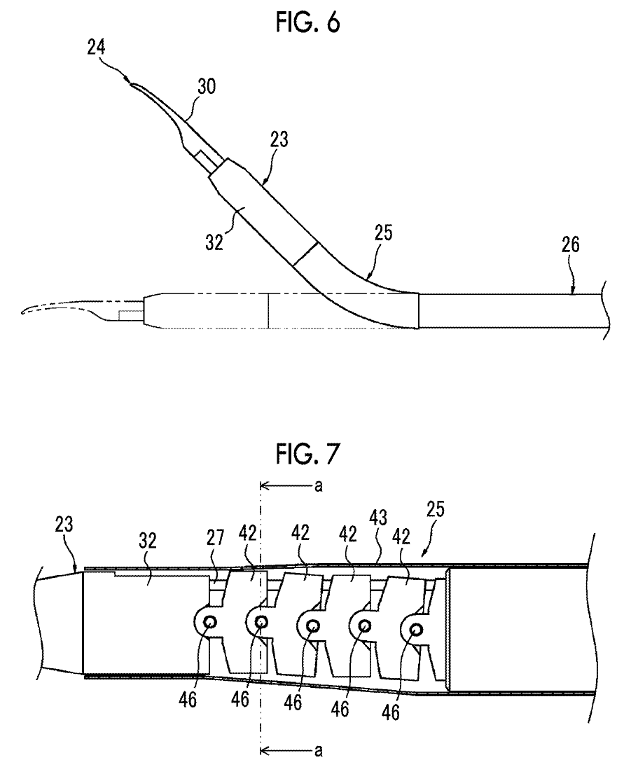

FIG. 5 is a diagram showing a configuration of the bendable part 25 and the connecting part 26 of the treatment tool for an endoscope 20. FIG. 6 is a diagram showing an operation of the bendable part 25 of FIG. 5.

As shown in FIG. 5, in the insertion part 21 of the treatment tool for an endoscope 20, the bendable part 25 is provided on the operation part 22 side of the distal end part 23 having the grip part 24, and the connecting part 26 is provided on the operation part 22 side of the bendable part 25. The connecting part 26 has flexibility and also has stiffness that allows translational and rotational power to be transmitted from the operation part 22 side to the bendable part 25 side. The connecting part 26 may be configured, for example, such that an outer periphery of a spiral tube, which is formed by spirally winding a strip material made of metal, is covered with a net-like tube formed by braiding a wire made of metal and an outer periphery of the net-like tube is covered with an outer coat made of resin.

As shown in FIG. 6, the bendable part 25 operated to be bent by the operation part 22 is bendable in a direction substantially perpendicular to a plane including an opening and closing direction of the pair of grip claws 30. A bending operation plane including a bending operation direction of the bendable part 25 is in a direction substantially perpendicular to an opening and closing operation plane of the pair of grip claws 30.

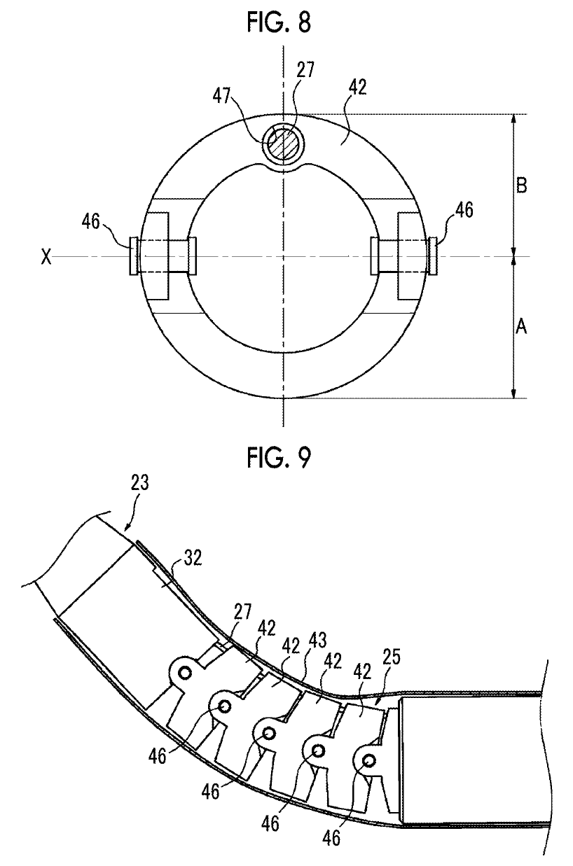

FIG. 7 is a diagram showing an internal configuration of the bendable part 25. FIG. 8 is a cross-sectional view taken along line a-a of the bendable part 25 shown in FIG. 7. FIG. 9 is a diagram showing an internal operation of the bendable part 25.

As shown in FIG. 7, the bendable part 25 has a plurality of bending pieces 42 and an outer coat 43 made of resin. The plurality of bending pieces 42 are arranged in a longitudinal direction of the insertion part 21 including the bendable part 25. Two bending pieces 42 adjacent to each other are connected to each other via a pair of pins 46 disposed to face each other in a radial direction of the bendable part 25.

As shown in FIG. 8, the pair of pins 46 are disposed on an axis X. The axis X is a direction substantially parallel to the opening and closing direction of the pair of grip claws 30. The adjacent bending pieces 42 connected by the pair of pins 46 are rotationally movable about the axis X as a rotational movement axis.

As shown in FIG. 9, the bendable part 25 is bent along a direction (bending operation plane) substantially perpendicular to the opening and closing direction (direction of the axis X) of the pair of grip claws 30 by adding up rotational movements of the plurality of bending pieces 42 with the axis X as the rotational movement axis.

The bendable part 25 is bent by the operation wire 27 for opening and closing the pair of grip claws 30. As shown in FIG. 8, each of the plurality of bending pieces 42 has a wire guide 47 that holds the operation wire 27 in a pushable and pullable manner. In a case where the bending piece 42 is divided into a first side A and a second side B, which is an opposite side, with the axis X as a boundary, the wire guide 47 is provided on one side, which is the second side B. Therefore, as the operation wire 27 is pulled to the operation part 22 side, the bendable part 25 is bent such that the first side A is positioned outside the curve and the second side B is positioned inside the curve.

As described above, in the treatment tool for an endoscope 20, the closing operation of the grip part 24 and the bending operation of the bendable part 25 are performed by pulling one operation wire 27. This makes it easier to operate the operation part 22. Here, in a case where the operation wire 27 is pulled to the operation part 22 side, the grip part 24 is first closed, and the bendable part 25 is bent in a state where the grip part 24 is closed. An operation sequence of the closing operation of the grip part 24 and the bending operation of the bendable part 25 can be set based on a magnitude relationship between an operation resistance in a case of closing the grip part 24 and an operation resistance in a case of bending the bendable part 25. For example, in a case where the operation resistance of the bendable part 25 is relatively large, the closing operation of the grip part 24 is performed first, and the bending operation of the bendable part 25 is performed later.

The operation resistance in a case where the grip part 24 is closed includes friction at the intersecting portion of the pair of grip claws 30 and friction at a connecting portion between the grip claw 30 and the link member 31. The operation resistance in a case where the bendable part 25 is bent includes friction at a connecting portion between the two adjacent bending pieces 42. In addition, the outer coat 43 of the bendable part 25 is an elastic member that linearly extends the bendable part 25. Therefore, the operation resistance in a case where the bendable part 25 is bent includes the elasticity of the outer coat 43. In addition, the operation wire 27 is also an elastic member that linearly extends the bendable part 25. Therefore, the operation resistance in a case where the bendable part 25 is bent includes the elasticity of the operation wire 27. The elastic member that extends the bendable part 25 linearly is not limited to the outer coat 43, the operation wire 27, and the like, and may be a wire spring, a leaf spring, or the like.

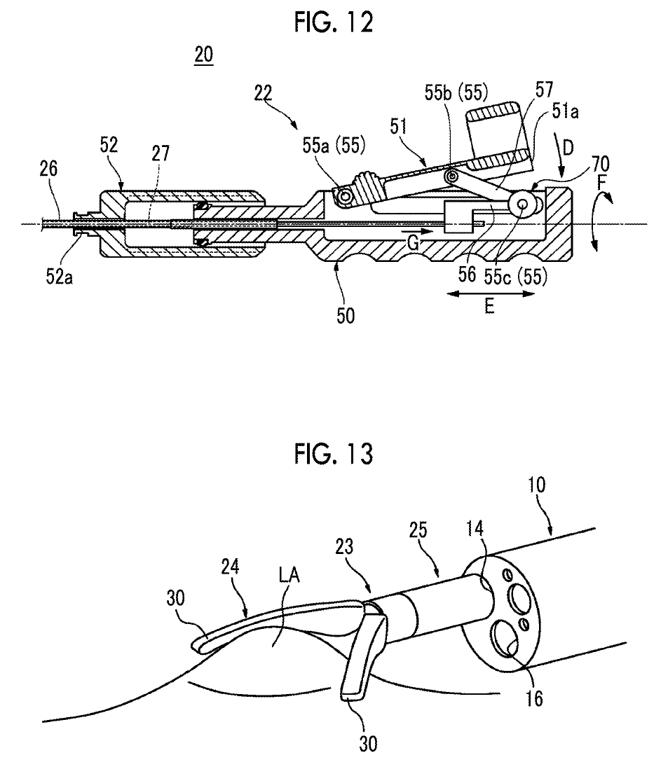

As described above, in the bendable part 25 that is bent by the pulling of the operation wire 27, as the outer coat 43 is made of an elastic member, an elastic force in a direction returning from a bent state to a linear state is generated by the outer coat 43. In addition, the elastic force increases according to the amount of bending of the bendable part 25. A friction adjustment mechanism 70 (described below in FIG. 12) is provided which can respond to a change in the elastic force, increase a frictional force according to the bending amount (angle) of the bendable part 25, and maintain the bent state of the bendable part 25 in a stable condition.

As the amount of movement by which the operation wire 27 is pulled increases, a force pulling in a direction of restoring the operation wire 27 (a linearly extending force) increases. In the present embodiment, the friction adjustment mechanism 70 to be described below, which can respond to a change in the force (elastic restoring force) pulling in the direction of restoring the operation wire 27, can increase a frictional force to correspond to the amount of pulling of the operation wire 27, and can maintain a pulled state in a stable condition.

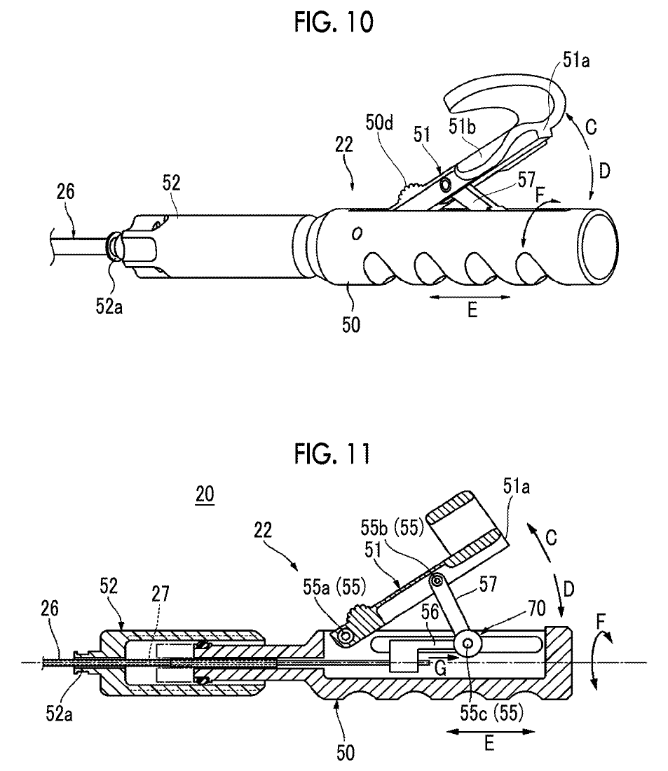

FIG. 10 is a diagram showing a configuration of the operation part 22 of the treatment tool for an endoscope 20. FIG. 11 is a cross-sectional view of the operation part 22 of the treatment tool for an endoscope 20.

As shown in FIG. 10, the operation part 22 includes an operation part main body 50, an operation handle 51, and a fixing part 52. The operation part main body 50 functions as means for inputting a forward and backward rotation operation of moving the grip part 24 (see FIG. 3) forward and backward and rotating the grip part 24. The operation handle 51 functions as means for inputting an opening and closing bending operation of opening and closing the grip part 24 and bending the bendable part 25 (see FIG. 2). The fixing part 52 is an attachment portion that is attachably and detachably attached to the endoscope operation part 7 (see FIG. 1).

The fixing part 52 has a connection metal fitting 52a. The connection metal fitting 52a is connected to a base provided at the first treatment tool insertion port 13 (see FIG. 1) of the endoscope operation part 7. The operation part 22 is supported by the endoscope operation part 7 in a state in which the connection metal fitting 52a is connected to the base.

The operation part main body 50 is formed in a rod shape and is operable in a central axis direction indicated by an arrow E and in a rotation direction around a central axis indicated by an arrow F. The operation part main body 50 is supported by the fixing part 52. The connecting part 26 of the insertion part 21 is connected to the operation part main body 50 through the fixing part 52. The connecting part 26 is moved forward and backward along a longitudinal axis of the connecting part 26 in response to the operation of the operation part main body 50 in the direction of the arrow E. In addition, the connecting part 26 is rotated about the longitudinal axis of the connecting part 26 in response to the operation of the operation part main body 50 in the direction of the arrow F. The forward and backward movement and rotation of the connecting part 26 are transmitted to the grip part 24, and the grip part 24 is also moved forward and backward and is rotated integrally with the connecting part 26.

The operation handle 51 is swingably supported by the operation part main body 50. For example, as shown in FIG. 11, the operation handle 51 is supported by a rotational movement support shaft part 55a (55), and can swing in an opening direction C in which a free end part 51a moves away from the operation part main body 50 and in a closing direction D approaching the operation part main body 50. The operation handle 51 is supported to move on a fan-shaped operation movement surface centered on the rotational movement support shaft part 55a.

FIG. 12 is a cross-sectional view showing a closed state of the operation part 22 of the treatment tool for an endoscope 20. The closed state of the operation part 22 is a state in which the operation handle 51 is operated in the closing direction D. Meanwhile, the open state of the operation part 22 is a state (FIG. 11) in a case where the operation handle 51 is operated in the opening direction C.

The operation handle 51 pulls (direction of an arrow G) the operation wire 27 in a case of swinging in the closing direction D and pushes (direction opposite to the arrow G) the operation wire 27 in a case of swinging in the opening direction C. The operation wire 27 is fixed to a wire holding part 56 that is provided to be slidably movable along its axis in the operation part main body 50. The wire holding part 56 is connected to the operation handle 51 via a link member 57 that is rotationally movably connected to the rotational movement support shaft part 55c (55). The link member 57 and the operation handle 51 are rotationally movably connected to each other via the rotational movement support shaft part 55b (55).

As described above, as the amount of pulling (the amount of movement in the direction of the arrow G) of the operation wire 27 increases, a force (elastic restoring force) pulling in a direction of restoring the wire holding part 56 increases. In addition, in a case where a lesion part is gripped and lifted, the restoring force increases with the lifting amount (the amount of pulling). Therefore, a restoring force acting on the operation handle 51 increases as the amount of pulling increases. In order to respond to a change in the restoring force, the friction adjustment mechanism 70 that increases a frictional force for locking the wire holding part 56 to correspond to the pulling amount of the operation wire 27 that is interlocked with an operation of the operation handle 51 is provided at the rotational movement support shaft part 55c.

The operation wire 27 is pushed out to the distal end part 23 side in response to the swinging of the operation handle 51 in the opening direction C and is pulled to the operation part 22 side in response to the swinging of the operation handle 51 in the closing direction D. In this case, as the operation handle 51 swings in the closing direction D, the grip part 24 is closed and the bendable part 25 is bent.

As the operation handle 51 has the friction adjustment mechanism 70 that maintains its operation state, the operation handle 51 is configured to be capable of maintaining a bending angle of the bendable part 25 at any one of angles that are equal to or smaller than a maximum bending angle. The friction adjustment mechanism 70 increases a frictional force for locking the wire holding part 56 to correspond to an increase in the pulling amount of the operation wire 27 caused by the wire holding part 56 that is interlocked with the swinging of the operation handle 51. Therefore, even in a case where the pulling amount of the operation wire 27 is increased and the wire restoring force is increased, the operation handle 51 is prevented from returning. Accordingly, a hand can be released from the operation handle 51 regardless of an operation position of the operation handle 51, and further, an operation force required for the rotational movement is small, so that a stable operation is possible.

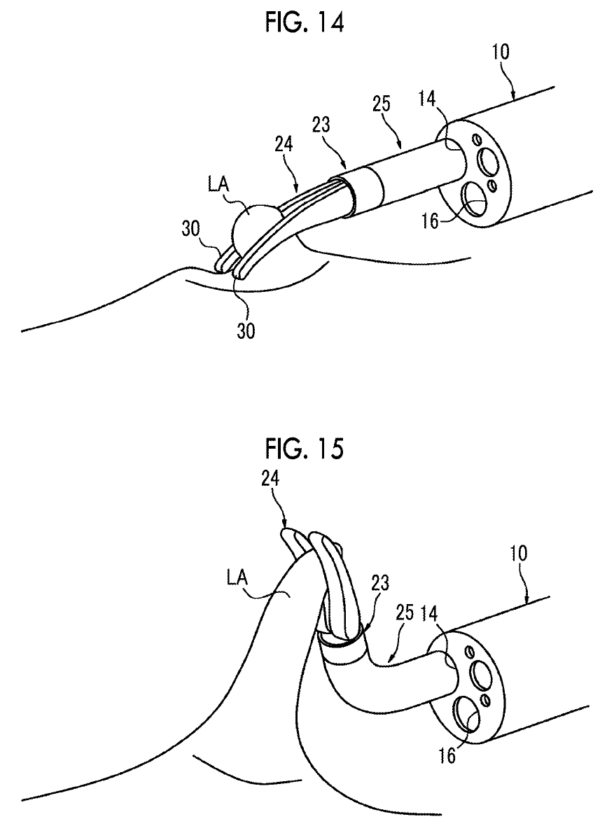

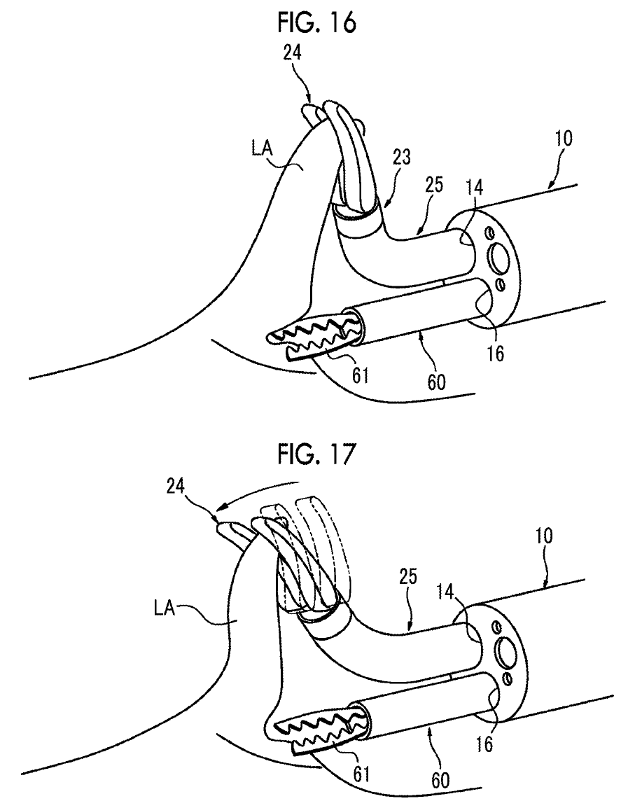

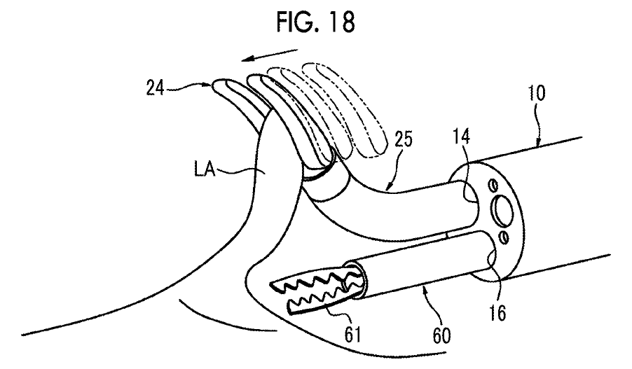

A first example of a treatment method using the treatment tool for an endoscope 20 will be described with reference to FIGS. 13 to 18. In this example, a treatment method of endoscopic submucosal dissection (ESD) will be described. FIG. 13 is a diagram showing a first example (part 1) of a treatment method using the treatment tool for an endoscope 20. FIG. 14 is a diagram showing a first example (part 2) of a treatment method using the treatment tool for an endoscope 20. FIG. 15 is a diagram showing a first example (part 3) of a treatment method using the treatment tool for an endoscope 20. In addition, FIG. 16 is a diagram showing a first example (part 1) of a treatment method using a combination of the treatment tool for an endoscope 20 and another treatment tool for an endoscope. FIG. 17 is a diagram showing a first example (part 2) of a treatment method using a combination of the treatment tool for an endoscope 20 and another treatment tool for an endoscope. FIG. 18 is a diagram showing a first example (part 3) of a treatment method using a combination of the treatment tool for an endoscope 20 and another treatment tool for an endoscope.

The operation wire 27 is pulled to the operation part 22 side in response to the swinging of the operation handle 51 in the closing direction D. In addition, another treatment tool for an endoscope used in combination with the treatment tool for an endoscope 20 is an incision tool and is high-frequency forceps 60 having a pair of openable and closable claws 61 at a distal end part (see FIG. 16). The pair of claws 61 are opened and closed by operating an operation part of the high-frequency forceps 60. In a state where the pair of claws 61 are closed and a living body tissue is gripped by the pair of claws 61, a high-frequency current flows between the pair of claws 61 and a return electrode plate via the living body tissue, or a high-frequency current flows between the pair of claws 61, thereby cauterizing and incising the living body tissue. The treatment tool for an endoscope 20 is an example of a “first treatment tool” of the present invention. The high-frequency forceps 60 are an example of a “second treatment tool” of the present invention.

As shown in FIG. 13, the endoscope distal end part 10 of the endoscope 2 inserted into the body is disposed on a side of a lesion part LA of a mucous membrane layer. The treatment tool for an endoscope 20 is inserted through the first treatment tool channel 14 of the endoscope 2, and the distal end part 23 and the bendable part 25 of the treatment tool for an endoscope 20 protrude from the end surface of the endoscope distal end part 10. Then, the lesion part LA is gripped by the grip part 24 of the distal end part 23 through the operation of the operation part 22 of the treatment tool for an endoscope 20.

In a case where the lesion part LA is gripped by the grip part 24, first, the operation handle 51 (see FIG. 10) of the operation part 22 is operated in the opening direction C. As shown in FIG. 13, by operating the operation handle 51 in the opening direction C, the operation wire 27 is pushed to the distal end part 23 side, and, by pushing the operation wire 27, the bendable part 25 is linearly extended and laid along the longitudinal axis of the connecting part 26. In addition, as the operation wire 27 is pushed out, the pair of grip claws 30 of the grip part 24 are opened. Then, the lesion part LA is disposed between the pair of grip claws 30 by appropriately pushing and pulling the operation part main body 50.

Next, the operation handle 51 is operated in the closing direction D in a state where the lesion part LA is disposed between the pair of grip claws 30. Accordingly, the operation wire 27 is pulled to the operation part 22 side. As the operation wire 27 is pulled, first, as shown in FIG. 14, the pair of grip claws 30 are closed and the lesion part LA is gripped by the grip part 24. Then, after the lesion part LA is gripped by the grip part 24, the bendable part 25 is bent as shown in FIG. 15. Accordingly, the grip part 24 is erected from a state of being laid along the longitudinal axis of the connecting part 26, and the lesion part LA gripped by the grip part 24 is lifted.

In a state where the lesion part LA is being lifted, the high-frequency forceps 60 inserted through the second treatment tool channel 16 of the endoscope 2 protrude from an edge surface of the endoscope distal end part 10 as shown in FIG. 16. The pair of claws 61 of the high-frequency forceps 60 are disposed at a lower part of the lesion part LA, and the lower part of the lesion part LA is incised by the pair of claws 61. As the incision proceeds, the lifted lesion part LA may be released once, re-gripped, and then lifted. In a case where the lesion part LA that has been incised once is lifted, the lower part can be exposed for easy viewing. The incision proceeds as the high-frequency forceps 60 is pushed and pulled as appropriate, and the lesion part LA including a submucosal layer is gradually peeled off.

In this way, the lesion part LA can be gripped from the side of the lesion part LA, and the gripped lesion part LA can be lifted by operating the operation handle 51 of the treatment tool for an endoscope 20, so that the operation is simple. In addition, by lifting the lesion part LA, the lower part of the lesion part LA can be exposed for easy viewing, and treatment for the lower part of the lifted lesion part LA can be performed safely, reliably, and easily.

In a case of incision, the operation part main body 50 of the operation part 22 may be pushed and pulled in the direction of the arrow E of FIG. 10 and/or the operation part main body 50 may be rotated in the direction of the arrow F of FIG. 10. As described above, the connecting part 26 has stiffness that allows translational and rotational power to be transmitted from the operation part 22 side to the bendable part 25 side, and pushing, pulling, and rotation of the operation part main body 50 are transmitted to the bendable part 25 via the connecting part 26.

FIG. 17 shows a movement of the grip part 24 in a case where the operation part main body 50 is rotated. The connecting part 26 is rotated about the longitudinal axis of the connecting part 26 in response to the rotation of the operation part main body 50. In a state in which the bendable part 25 is bent, the grip part 24 is rotated while maintaining a state of being erected with respect to the longitudinal axis of the connecting part 26. As the grip part 24 is rotated, the lesion part LA gripped by the grip part 24 is swung around the longitudinal axis of the connecting part 26.

FIG. 18 shows a movement of the grip part 24 in a case where the operation part main body 50 is pushed and pulled. The connecting part 26 is moved forward and backward in an axial direction of the longitudinal axis of the connecting part 26 in response to the pushing and pulling of the operation part main body 50. In a state in which the bendable part 25 is bent, the grip part 24 is moved forward and backward while maintaining a state of being erected with respect to the longitudinal axis of the connecting part 26. As the grip part 24 is moved forward and backward, the lesion part LA gripped by the grip part 24 is pushed and pulled in the axial direction of the longitudinal axis of the connecting part 26.

By swinging and/or pushing and pulling the lesion part LA as appropriate, for example, an incised wound can be widened. Accordingly, treatment for the lower part of the lesion part LA can be performed more easily.

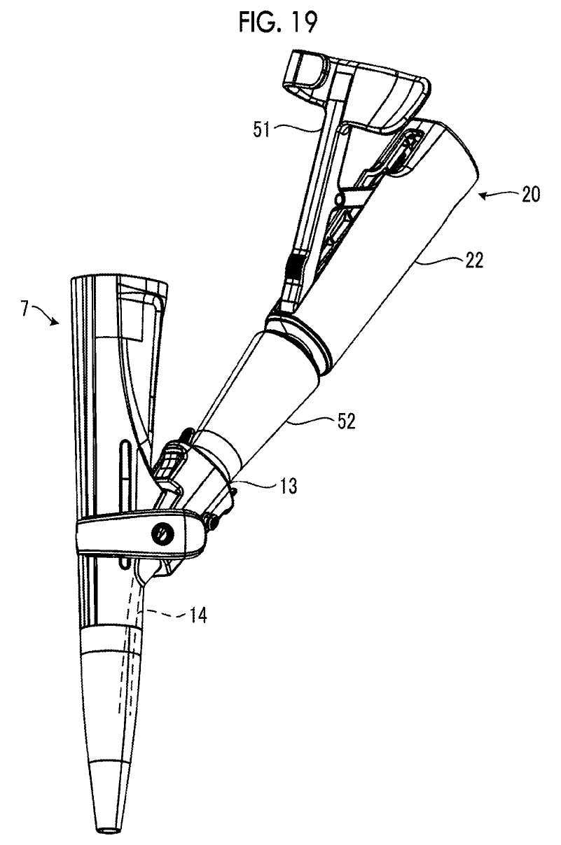

FIG. 19 is a diagram showing a state in which the treatment tool for an endoscope 20 is attached to the first treatment tool insertion port 13 of the endoscope operation part 7. As described above, the treatment tool for an endoscope 20 is attached to the endoscope operation part 7 by connecting the connection metal fitting 52a of the fixing part 52 to the base of the first treatment tool insertion port 13 of the endoscope operation part 7. In the treatment tool for an endoscope 20 attached to the endoscope operation part 7, the insertion part 21 consisting of the distal end part 23, the bendable part 25, and the connecting part 26 is inserted into the first treatment tool channel 14 that reaches the endoscope distal end part 10 from the first treatment tool insertion port 13.

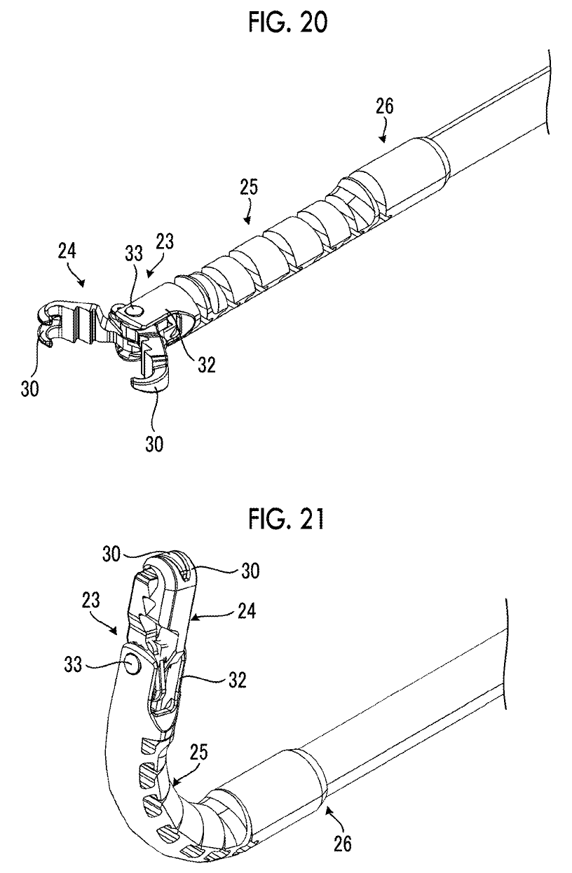

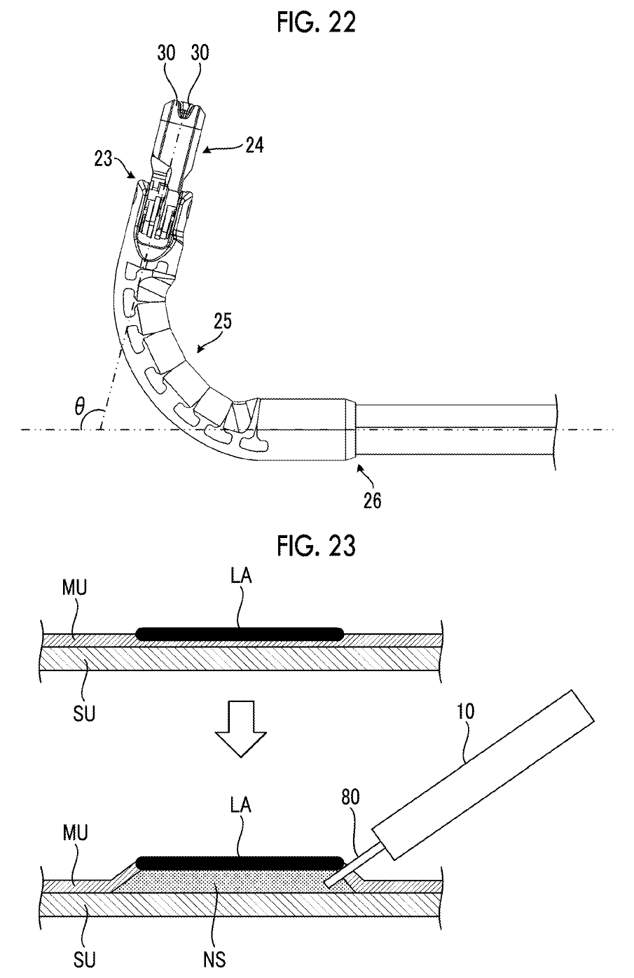

The bent state of the bendable part 25 in the insertion part 21 of the treatment tool for an endoscope 20 will be described with reference to FIGS. 20 to 22. FIG. 20 is a diagram showing the bendable part 25 in the insertion part 21 in a non-bent state. FIG. 21 is a perspective view showing the bendable part 25 in a bent state. FIG. 22 is a plan view of the bendable part 25 shown in FIG. 21.

As shown in FIG. 20, the non-bent state of the bendable part 25 means a state in which the bendable part 25 is straight with respect to an end part of the connecting part 26 on the bendable part 25 side. As described above, the bendable part 25 is bent by the operation of the operation wire 27 that reaches the operation part 22 from the distal end part 23 through the bendable part 25 and the connecting part 26. In addition, the pair of grip claws 30 at the distal end part 23 are opened and closed by the operation of the operation wire 27.

For example, in a case where the operation wire 27 is pushed out to the distal end part 23 side, as shown in FIG. 20, the bendable part 25 is brought into a non-bent state where the bendable part25 is linearly extended and laid along the longitudinal axis of the connecting part 26, and the pair of grip claws 30 of the grip part 24 are in an open state.

On the other hand, in a case where the operation wire 27 is pulled to the operation part 22 side, as shown in FIGS. 21 and 22, the pair of grip claws 30 of the grip part 24 are brought into a closed state, and the bendable part 25 is bent so as to bulge and is brought into an upwardly bent state with respect to the longitudinal axis of the connecting part 26. In this case, in a case where the operation wire 27 is pulled to the operation part 22 side, first, the pair of grip claws 30 are brought into a closed state, and, after the pair of grip claws 30 are closed, the bendable part 25 is brought into a bent state.

As shown in FIG. 22, a maximum bending angle θ of the bendable part 25 with respect to the longitudinal axis of the connecting part 26 is configured to be greater than 90 degrees. The expression “maximum bending angle” refers to an angle of a direction of the distal end of the bendable part 25 in a case where the bendable part 25 is maximally bent relative to a direction of the distal end of the bendable part 25 in a case where the bendable part 25 is in a non-bent state. The expression “maximally bent” refers to a state in which the bendable part 25 is bent to the greatest extent within a range that can be operated by operating the operation part 22. The maximum bending angle θ is preferably, for example, 100 degrees to 180 degrees.

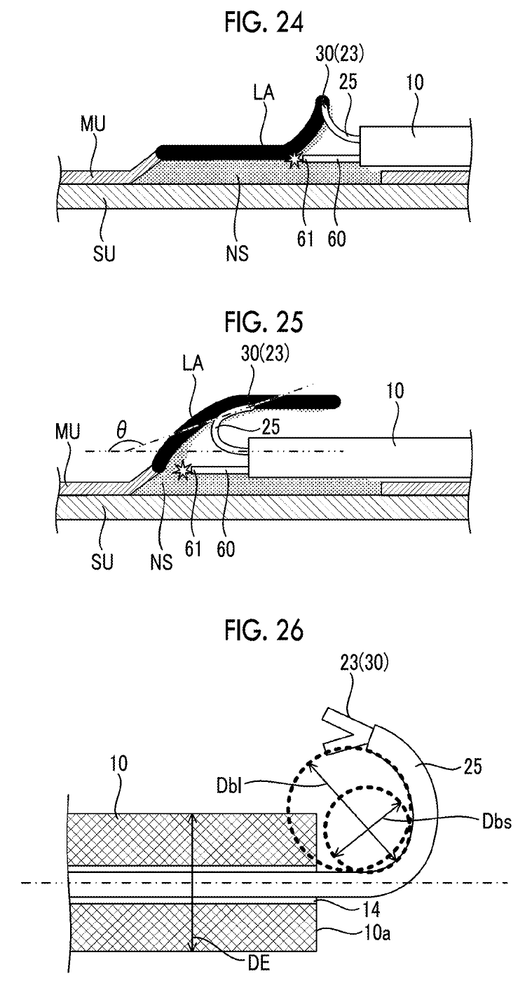

A second example of a treatment method of the ESD using the treatment tool for an endoscope 20 will be described with reference to FIGS. 23 to 25. FIG. 23 is a diagram showing a second example (part 1) of a treatment method using the treatment tool for an endoscope 20. FIG. 24 is a diagram showing a second example (part 2) of a treatment method using the treatment tool for an endoscope 20. FIG. 25 is a diagram showing a second example (part 3) of a treatment method using the treatment tool for an endoscope 20.

As shown in FIG. 23, an injection needle 80 protruding from the end surface of the endoscope distal end part 10 is used to inject a drug (for example, physioligical saline) into a submucosal layer SU, thereby lifting the lesion part LA together with a mucous membrane MU. The injection needle 80 is inserted through the second treatment tool channel 16 of the endoscope 2.

Next, the mucous membrane MU around the lifted lesion part LA is incised. The lesion part LA is gradually peeled off from the incision part. For example, as shown in FIG. 24, the distal end part 23 and the bendable part 25 of the treatment tool for an endoscope 20 inserted through the first treatment tool channel 14 of the endoscope 2 and the high-frequency forceps 60 inserted through the second treatment tool channel 16 protrude from the end surface of the endoscope distal end part 10. After the lesion part LA is gripped by the pair of grip claws 30 of the distal end part 23 of the treatment tool for an endoscope 20, the bendable part 25 is bent to lift the lesion part LA gripped by the grip claws 30. Then, in a state where the lesion part LA is lifted, the lower part of the lesion part LA is incised by the pair of claws 61 of the high-frequency forceps 60.

In incising the lesion part LA, in a case where the lesion part LA to be incised is small in size or in a case where the incision of the lesion part LA is at its initial stage, the lifting amount (distance) required to lift the lesion part LA gripped by the grip claws 30 is small. Therefore, for example, as shown in FIG. 24, a direction of the pulling force of the bendable part 25 generated by bending the bendable part 25 is a direction (upward direction in FIG. 24) in which the gripped lesion part LA is pulled away from the submucosal layer SU. Therefore, a bending angle of the bendable part 25 required to lift the lesion part LA may be 90 degrees or less.

However, in a case where the lesion part LA to be incised is large in size or in a case where the incision of the lesion part LA has already progressed to a certain extent, the lifting amount (distance) required to lift the lesion part LA gripped by the grip claws 30 becomes large. Therefore, for example, as shown in FIG. 25, a direction of the pulling force of the bendable part 25 generated by bending the bendable part 25 is a direction in which the gripped lesion part LA is pulled away from the submucosal layer SU and is also a direction (right direction in FIG. 25) in which the gripped lesion part LA is sent in a direction opposite to an incision direction. Therefore, the maximum bending angle θ of the bendable part 25 for lifting the lesion part LA and then sending the lifted lesion part LA in the direction opposite to the incision direction needs to be an angle greater than 90 degrees.

FIG. 26 is a diagram showing a positional relationship between the distal end part 23 and the endoscope 2 in a case where the bendable part 25 is maximally bent. As shown in FIG. 26, the bendable part 25 and the distal end part 23 of the treatment tool for an endoscope 20 inserted through the first treatment tool channel 14 of the endoscope 2 extend outward from an end surface 10a of the endoscope distal end part 10. In a case where the extended bendable part 25 is bent to the maximum bending angle (in a case of being maximally bent), an inner diameter of an arc drawn by the bendable part 25 is greater than 1/2 of an outer diameter DE of the endoscope distal end part 10.

The “inner diameter of the arc” is twice the curvature radius. In a case where the curvature is not constant, the curvature radius is twice the curvature radius of a portion having the highest curvature (large bending). For example, as in this example, in a case where the curvature of the arc drawn by the bendable part 25 is not constant, an inner diameter Dbs is twice the curvature radius of the portion having the highest curvature (large bending), and an inner diameter Dbl is twice the curvature radius of a portion having the lowest curvature (small bending), the inner diameter of the arc drawn by the bendable part 25 is the inner diameter Dbs. Since a shape of a portion of the endoscope 2 in which the first treatment tool channel 14 is provided is substantially circular as viewed along a longitudinal axis of the first treatment tool channel 14, the “outer diameter DE of the endoscope distal end part 10” is an outer diameter (diameter) of the portion. In a case where a shape of the endoscope distal end part 10 is elliptical, a diameter of a major axis of the ellipse is the outer diameter DE. Therefore, a relationship between the inner diameter Dbs of the arc drawn by the bendable part 25 and the outer diameter DE of the endoscope distal end part 10 is Dbs > DE/2.

In addition, the bendable part 25 is configured such that the distal end part 23 (gripping claw 30) extending from the first treatment tool channel 14 does not come into contact with the endoscope distal end part 10 of the endoscope 2 in a case where the bendable part 25 is maximally bent. That is, the bendable part 25 is not in contact with the endoscope distal end part 10 regardless of the bent state in which the bending angle is changed. Furthermore, in a case where the bendable part 25 is maximally bent, the distal end part 23 extending from the first treatment tool channel 14 is not in contact with the endoscope distal end part 10 of the endoscope 2 regardless of the rotation state of the insertion part 21 accompanying the operation of the operation part main body 50.

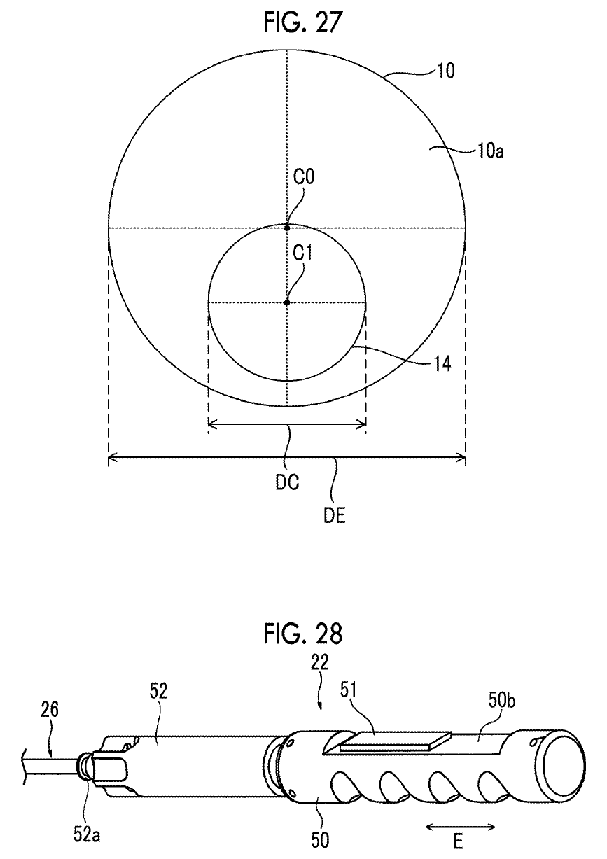

FIG. 27 is a diagram showing an example of the end surface 10a of the endoscope distal end part 10. As shown in FIG. 27, the first treatment tool channel 14 of the endoscope 2 is provided such that a position C1 at the center of the first treatment tool channel 14 is a position deviated from a position C0 at the center of the endoscope distal end part 10 of the endoscope 2. Although not shown in FIG. 27, in addition to the first treatment tool channel 14, the second treatment tool channel 16 and the like are formed in the endoscope distal end part 10 of the endoscope 2 (see, for example, FIG. 13), and the second treatment tool channel 16 is also provided such that a position at the center of the second treatment tool channel 16 is deviated from the position C0 at the center of the endoscope distal end part 10 of the endoscope 2. The outer diameter DE of the endoscope distal end part 10 is, for example, 5 mm to 14 mm, and preferably 9 mm to 12 mm. An inner diameter DC of the first treatment tool channel 14 is, for example, 2 mm to 6 mm, and preferably 2.8 mm to 3.8 mm.

As described above, in the treatment tool for an endoscope 20, the pair of grip claws 30 of the grip part 24 can be closed by the operation of the operation handle 51, the bendable part 25 can be bent in a state where the grip claws 30 are closed, and the maximum bending angle of the bent bendable part 25 can be set to be greater than 90 degrees. With this configuration, by bending the bendable part 25, it is possible to continuously pull the lesion part LA gripped by the grip claws 30 over a wide range. Therefore, for example, even in a case where the lesion part LA to be incised is a large part, or even in a case where the incision process of the lesion part LA has progressed to a certain extent, the number of times the lesion part LA needs to be re-gripped by the grip claws 30 can be reduced. As a result, the lower part of the lesion part LA can be exposed for easy viewing, and treatment for the lower part of the lesion part LA can be performed safely, reliably, and easily. Therefore, it is possible to improve the ease of treatment on the lesion part LA.

In addition, in the treatment tool for an endoscope 20, in a case where the bendable part 25 is maximally bent, the inner diameter Dbs of the arc drawn by the bendable part 25 is greater than 1/2 of the outer diameter DE of the endoscope distal end part 10. Therefore, even in a case where the bendable part 25 is maximally bent, the distal end part 23 extending from the first treatment tool channel 14 does not come into contact with the endoscope distal end part 10 of the endoscope 2. In addition, even in a case where the bendable part 25 is maximally bent, the distal end part 23 extending from the first treatment tool channel 14 does not come into contact with the endoscope distal end part 10 regardless of the rotation state of the insertion part 21. Accordingly, in a case where the lesion part LA is gripped and lifted by the grip claws 30, it is possible to safely and reliably perform treatment on the lower part of the lesion part LA. Therefore, it is possible to improve the ease of treatment on the lesion part LA.

In the operation part 22 of the above embodiment, the operation handle 51 is provided to protrude from the operation part main body 50 and to be swingable, and the wire holding part 56 is moved by the swing, but the present invention is not limited to this configuration. FIG. 28 is a diagram showing another example of the operation handle 51. For example, as shown in FIG. 28, the operation handle 51 may be a member that is provided on a plane portion 50b provided at the operation part main body 50 and that is movable in a direction E along the plane portion 50b. The operation handle 51 is operated, for example, by a thumb of an operator gripping the operation part main body 50.

In this case, the operation handle 51 is connected to the wire holding part 56, and, in a case where the operation handle 51 is moved to a distal end side (an upper side of FIG. 28) of the operation part main body 50, the wire holding part 56 is also moved to the distal end side of the operation part main body 50, and the operation wire 27 is pulled. In addition, in a case where the operation handle 51 is moved to a base end side (a lower side of FIG. 28) of the operation part main body 50, the wire holding part 56 is also moved to the base end side of the operation part main body 50, and the operation wire 27 is loosened. That is, in the configuration shown in FIG. 28, through an operation of moving the operation handle 51 up and down, the same operation as an operation of swinging the operation handle 51 shown in FIG. 11 or the like can be realized. In the configuration shown in FIG. 28, the position or shape of the operation handle 51 or the plane portion 50b is not limited to the configuration shown in FIG. 28.

### Explanation of References

1: endoscope system

2: endoscope

3: light source device

4: processor

5: monitor

6: endoscope insertion part

7: endoscope operation part

8: universal cord

9: connector

10: endoscope distal end part

10a: end surface

11: endoscope bendable part

12: endoscope connecting part

13: first treatment tool insertion port

14: first treatment tool channel

15: second treatment tool insertion port

16: second treatment tool channel

20: treatment tool for endoscope

21: insertion part

22: operation part

23: distal end part

24: grip part

25: bendable part

26: connecting part

27: operation wire

30: grip claw

31, 57: link member

32: support

33, 46: pin

42: bending piece

43: outer coat

47: wire guide

50: operation part main body

50b: plane portion

51: operation handle

51a: free end part

52: fixing part

52a: connection metal fitting

55a, 55b, 55c: rotational movement support shaft part

56: wire holding part

60: forceps

61: claw

70: friction adjustment mechanism

80: injection needle

C0, C1: position

What is claimed is:

1. A treatment tool for an endoscope, comprising:
an insertion part that is insertable into a body; and
an operation part that is disposed on a base end side of the insertion part,
wherein the insertion part includes
a distal end part having a grip part that is openable and closable,
a bendable part that is bendable and that is provided to be adjacent to the distal end part on a side of the operation part, and
a connecting part that connects the bendable part and the operation part to each other,
the insertion part is capable of being moved forward and backward along a longitudinal axis of the connecting part and being rotated about the longitudinal axis by an operation of the operation part,
by an operation of the operation part, the grip part is closed and the bendable part is bent in a state where the grip part is closed, and
a maximum bending angle of the bendable part with respect to the longitudinal axis is greater than 90 degrees.

2. The treatment tool for an endoscope according to claim 1, further comprising:
at least one operation wire that is connected to the operation part and that operates at least one of opening and closing of the grip part or bending of the bendable part.

3. The treatment tool for an endoscope according to claim 1,
wherein the insertion part is used by being inserted into a treatment tool channel of the endoscope, and
an inner diameter of an arc drawn in a case where the bendable part is maximally bent is greater than 1/2 of an outer diameter of the endoscope.

4. The treatment tool for an endoscope according to claim 1,
wherein the insertion part is used by being inserted into a treatment tool channel of the endoscope, and
in a case where the bendable part is maximally bent, the distal end part extending from the treatment tool channel is not in contact with the endoscope.

5. The treatment tool for an endoscope according to claim 4,
wherein, in a case where the bendable part is maximally bent, the distal end part extending from the treatment tool channel is not in contact with the endoscope regardless of a rotation state of the insertion part.

6. An endoscope system comprising:
the treatment tool for an endoscope according to claim 1; and
an endoscope having a treatment tool channel through which the insertion part is insertable.

7. The endoscope system according to claim 6, further comprising:
a first treatment tool that is the treatment tool for an endoscope;
a second treatment tool; and
the endoscope,
wherein the endoscope has a first treatment tool channel through which the insertion part of the first treatment tool is insertable and a second treatment tool channel through which the second treatment tool is insertable.
